(19) United States
(12) Patent Application Publication
(10) Pub. No.: US 2026/0232260 A1
(43) Pub. Date: Aug. 13, 2026

(54) NON-INVASIVE DETECTION OF FRACTURE AND/OR FATIGUE OF A BONE

(71) Applicant: The Johns Hopkins University, Baltimore, MD (US)

(72) Inventors: Anna E. Knight, Silver Spring, MD (US); Eyal Bar-Kochba, Clarksville, MD (US); Nadeau J. Hahne, Ellicott City, MD (US); Constantine K. Demetropoulos, Bethesda, MD (US); Connor O. Pyles, Columbia, MD (US); Catherine M. Carneal, Silver Spring, MD (US)

(21) Appl. No.: 19/232,568

(22) Filed: Jun. 9, 2025

Related U.S. Application Data
(60) Provisional application No. 63/755,885, filed on Feb. 7, 2025.

(51) Int. Cl. A61B 5/00

(52) U.S. Cl. CPC A61B 5/4504; A61B 5/0095

(57) ABSTRACT

A system for detecting fracture or fatigue in a bone of a subject, the system comprising an ultrasound transmitter capable of being located exterior to the subject, an ultrasound receiver capable of being located exterior to the subject, and electronic circuitry to perform a method, the method comprising sending a first signal to the ultrasound transmitter, the first signal configured to cause the ultrasound transmitter to transmit a first ultrasound signal, receiving a second signal from the ultrasound receiver, the ultrasound receiver producing the second signal in response to a second ultrasound signal received by the ultrasound receiver, and processing the first signal and the second signal to detect at least one of fracture or fatigue in the bone of the subject.

### CROSS-REFERENCE TO RELATED APPLICATIONS

This application claims priority to U.S. Provisional Patent Application No. 63/755,885, filed Feb. 7, 2025, which is incorporated herein by reference in its entirety.

### BACKGROUND

Injuries such as stress fractures or bone fatigue are common injuries during physical activities such as running. Fractures and fatigue in bones are commonly detected through a number of conventional techniques, such as X-rays. Certain types of minor fractures and fatigue (e.g., stress fractures), however, cannot be detected or may be difficult to detect—and therefore, treated or prevented—by conventional methods until they grow larger, thereby causing greater injury to the patient. Moreover, conventional detection techniques can be time-consuming and costly, thereby discouraging regular, preventative usage by the patient. In addition, too much exposure to X-rays, which are used in conventional techniques, can have harmful effects on patients.

### SUMMARY OF THE INVENTION

According to one aspect of the disclosure, a system for detecting fracture or fatigue in a bone of a subject is provided. The system can include an ultrasound transmitter, an ultrasound receiver, and electronic circuitry. The ultrasound transmitter can be capable of being located exterior to the subject. The ultrasound receiver can be capable of being located exterior to the subject. The electronic circuitry can perform a method including, sending a first signal to the ultrasound transmitter, the first signal configured to cause the ultrasound transmitter to transmit a first ultrasound signal; receiving a second signal from the ultrasound receiver, the ultrasound receiver producing the second signal in response to a second ultrasound signal received by the ultrasound receiver; and processing the first signal and the second signal to detect fracture or fatigue in the bone of the subject.

According to another aspect of the disclosure, an apparatus for detecting fracture or fatigue in a bone of a subject is provided. The apparatus can include an ultrasound transmitter, an ultrasound mount, and an ultrasound receiver. The ultrasound transmitter can be configured to transmit a first ultrasound signal. The ultrasound mount can have contact surface, a mounting surface, and a transmitter holder, wherein: the contact surface is configured to contact a surface of the subject; the mounting surface is angled at an angle to a transverse direction of the bone and not parallel to the transverse direction of the bone; and the transmitter holder is configured to hold the ultrasound transmitter proximate the subject and at the angle. The ultrasound receiver can be configured to receive a second ultrasound signal and positioned at a distance from the ultrasound transmitter.

According to another aspect of the disclosure, a method for detecting fracture or fatigue in a bone of a subject is provided. The method can include positioning an ultrasound transmitter proximate to the subject, exterior to the subject, and above the bone of the subject. The ultrasound transmitter can be positioned at an angle to a transverse direction of the bone and not parallel to the transverse direction of the bone. The method can further include positioning an ultrasound receiver proximate the subject, exterior to the subject, and above the bone of the subject, the ultrasound receiver being positioned a distance from the ultrasound transmitter along the transverse direction of the bone; transmitting, from the ultrasound transmitter, first ultrasound signal; receiving, at the ultrasound receiver, second ultrasound signal; and detecting fracture or fatigue in the bone of the subject based on the second ultrasound signal.

These and other advantages and features will become more apparent from the following description taken in conjunction with the drawings.

### BRIEF DESCRIPTION OF DRAWINGS

FIG. 1 depicts a schematic overview of an exemplary system for detecting fracture or fatigue in a bone of a subject according to some embodiments.

FIG. 2 depicts a schematic overview of electronic circuitry according to some embodiments.

FIGS. 3-4 are flowcharts of exemplary methods for detecting fracture or fatigue in a bone of a subject according to some embodiments.

FIGS. 5A-5E depict exemplary apparatuses for detecting fracture or fatigue in a bone of a subject according to some embodiments.

FIGS. 6A-6B depict an experimental test setup for measuring transmitted and received signals in a healthy bone, and the results of the measurements.

FIGS. 7A-7B depict an experimental test setup for measuring delay time in a fractured bone, and the results of the experiment.

FIGS. 8A-8F depict an experimental test setup for measuring coherence and delay time as a result of the number of fatigue cycles in a bone, and the results of the experiment, as well as validation with microCT images.

### DETAILED DESCRIPTION OF THE EMBODIMENTS

As recognized by the inventors, it may be desirous to develop systems and methods which can detect stress fractures and/or fatigue in bone (collectively and hereinafter, “stress fractures”) which may be too small to be detected by conventional techniques, which may use X-rays. Such stress fractures may be caused by physical activity, such as running or distance walking, and/or by inadequate footwear. As recognized by the inventors, early detection of stress fractures can allow patients to receive treatment and/or intervention more quickly, before any minor injury further develops into a greater injury requiring a longer recovery period and potentially more expensive health care. Moreover, such a detection technique that does not employ X-rays may be beneficial to the overall health of the patient due to not requiring the use of X-rays on the patient.

As discovered by the inventors, one or more embodiments disclosed herein may serve as a solution for detecting minor injuries in bone. In certain industries, such as the military and athletics, running and other physical activities—and the injuries caused thereby—are common experiences for participants. For example, recent studies have shown that up to 8% of military recruits experience stress fractures as a result of frequent running, in part due to heavy gear loads and new shoes and/or boots which have yet to be “broken in.” See P. Takkar et al., “Stress Fractures in Military Recruits: A Prospective Study for Evaluation of Incidence, Patterns of Injury and Invalidments Out of Service,” Medical Journal, Armed Forces India, 75(3), 330-34 (Feb. 23, 2019). Studies also show that up to 87% of stress fractures occur in the tibias of recruits, resulting in an average rehabilitation time of approximately 21 weeks. See id.; A. M. Wood et al., “Incidence and Time to Return to Training for Stress Fractures during Military Basic Training,” Journal of Sports Medicine (Hindawi Publ. Corp.) (Jan. 21, 2014). Finally, recruits who experience stress fractures are four times more likely to be discharged than recruits who do not. See R. R. Protzman et al., “Stress Fractures in Men and Women Undergoing Military Training,” The Journal of Bone and Joint Surgery, American Volume, 59(6), 825 (September 1997). For track and field athletes, 21% sustain a stress fracture, with 46% of stress fractures being in the tibia. See K. L. Bennell et al., “The Incidence and Distribution of Stress Fractures in Competitive Track and Field Athletes. A Twelve-Month Prospective Study,” Am J Sports Med., 24(2):211-217 (March-April 1996).

As explained above, conventional techniques for detecting stress fractures are not sensitive enough to detect such fractures in their early stages, thereby worsening the injury. Conventional techniques are also impractical because they cannot be performed by users in the field on an as-needed basis. Accordingly, the inventors have discovered a need for systems and methods which are capable of the early detection of stress fractures by users (e.g., health care workers, physical trainers, athletic trainers, front line care providers, etc.), and in a convenient fashion.

Broadly, the inventors have discovered that stress fractures can be detected by inducing surface acoustic waves (SAWs) in a patient's bone and electronically processing associated data. SAWs may be considered a type of stress wave which are bound to the surface of a solid and attenuate less than, and move at a slower velocity than, bulk acoustic waves, which may be known as compression or shear waves. More specifically, the inventors have discovered that by placing an ultrasound transmitter on the shin of a user's leg at a particular angle (or range of angles), and by transmitting an ultrasound signal into the user's leg at a particular frequency (or range of frequencies), SAWs can be induced in the cortical surface of the user's tibia. These SAWs can be received by an ultrasound receiver which is also positioned on the user's shin at a certain distance from the ultrasound transmitter. By detecting a change in one or more measured parameters (such as the frequency profile of the received ultrasonic wave (e.g., by determining a coherence of an input signal to the ultrasound transmitter and an output signal from the ultrasound receiver)), a non-invasive determination can be made as to whether a stress fracture is present in the tibia. With the discovered techniques, detecting stress fractures in a bone of a subject can be determined non-invasively since the device to detect stress fractures is applied to the exterior of the subject and since no punctures, pricks, cuts, or openings in the subject are required. Moreover, with the discovered techniques, detecting stress fractures in a bone of a subject can be determined without the use of X-rays, as in conventional techniques, thereby promoting the health of the subject.

In this way, the inventors have discovered a practical application of SAWs to solve a number of technical problems inherent to conventional stress fracture detection techniques. For example, by positioning the ultrasound transmitter within an ergonomically-shaped mount or housing, a handheld device for detecting stress fractures in a bone can be achieved. In some embodiments, the device can be elaborated on by also integrating the ultrasound receiver therein. By using such a handheld device, a military recruit, an athlete, or a supervisor, coach, or health care provider of such a person can apply the device to the potentially-fractured bone to instantly detect whether the patient has a stress fracture, on demand. By contrast, conventional techniques require the patient to attend a physician's office or imaging office—which may not always be practical, convenient, or feasible—and then wait several days to receive results; and as explained above, such conventional techniques may not even be capable of detecting such minor stress fractures.

As discovered by the inventors, stress fractures may be detected in a bone of a subject using the techniques disclosed herein. In discussing various embodiments herein, the bone of the subject may be the tibia of a subject. While the tibia is used as an example bone to illustrate the invention, other bones of the subject may also be non-invasively inspected using the techniques disclosed herein. For example, other bones that may also be non-invasively inspected using the techniques disclosed herein include metatarsal, radius, ulna, clavicle, metacarpals, phalanges, rib, and calcaneus bones.

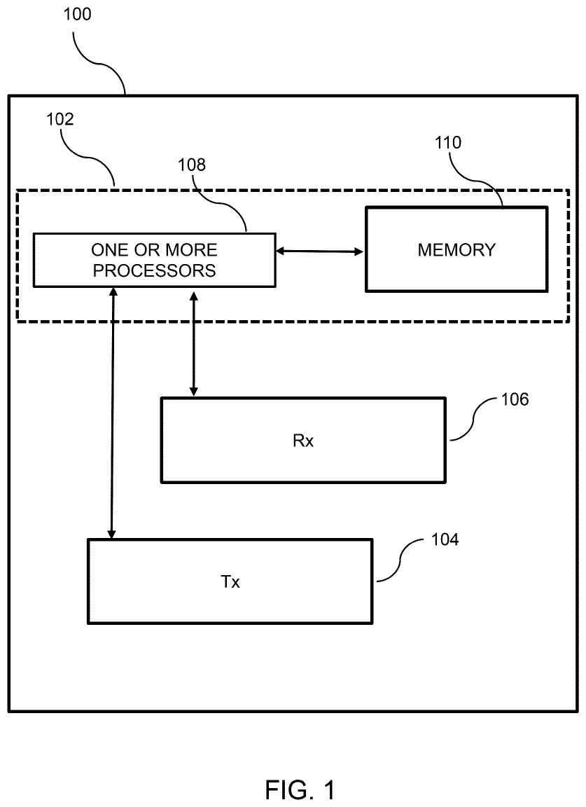

FIG. 1 depicts a schematic overview of an exemplary system for detecting stress fractures in a bone of a subject. System 100 can include electronic circuitry 102, an ultrasound transmitter 104, and an ultrasound receiver 106. The ultrasound transmitter and receiver 104, 106 can each be commercially-available, off-the-shelf components. In some embodiments, the electronic circuitry 102 can be capable of sending a first electrical signal to the ultrasound transmitter 104 which causes the ultrasound transmitter 104 to transmit a first ultrasound signal. The electronic circuitry 102 can also enable the ultrasound receiver 106, upon receiving an ultrasound signal, to produce a second electrical signal. In some embodiments, and as will be explained in further detail below, the ultrasound signal received by the ultrasound receiver 106 can be a second ultrasound signal which is different from the first ultrasound signal transmitted by the ultrasound transmitter 104. As will also be explained in further detail below, in some embodiments the electronic circuitry 102 can process the first electrical signal and the second electrical signal to detect stress fractures in the bone of a subject patient. In some embodiments, the electronic circuitry 102 may include one or more processors 108 and memory 110 to perform various techniques disclosed herein. In some embodiments, the electronic circuitry 102 may include dedicated electronic circuitry (e.g., customized digital signal processing circuits) to perform various techniques disclosed herein.

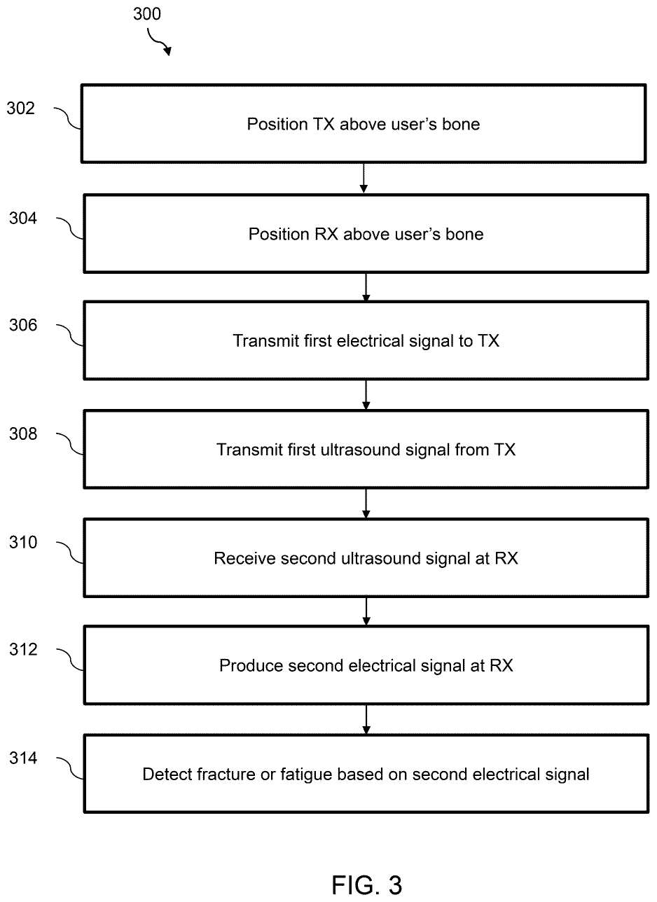

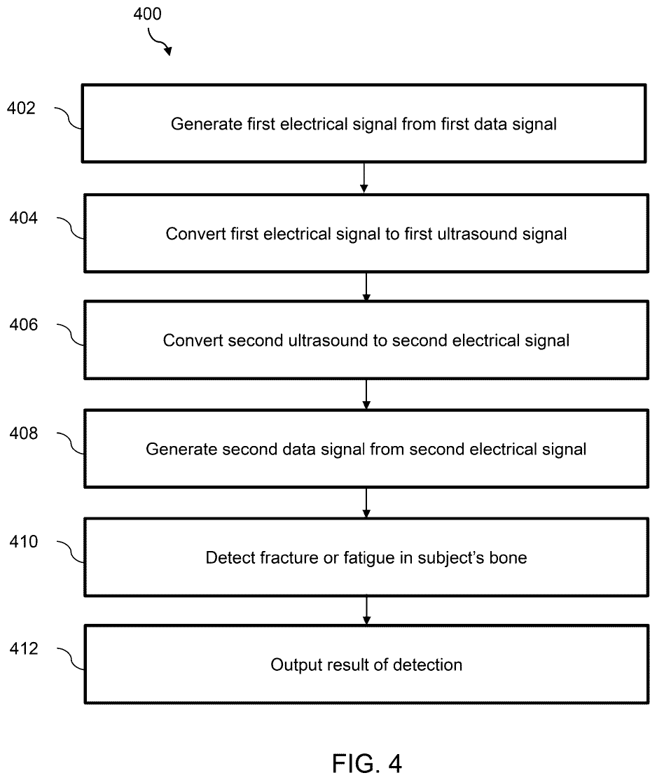

As an example, the electronic circuitry 102 may be a computer to implement certain inventive techniques disclosed herein. As an example, some or all of the steps in the method illustrated in FIG. 3 and/or FIG. 4 may be performed using a single apparatus having electronic circuitry 102. As an example, the steps in the method illustrated in FIG. 3 and/or FIG. 4 may be performed by one, two, three, four, or more apparatuses having electronic circuitry 102.

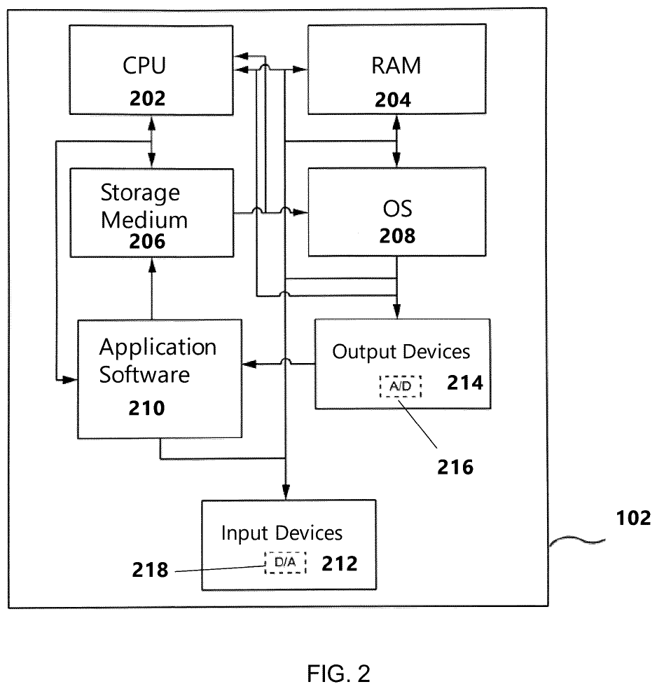

FIG. 2 depicts a schematic overview of an exemplary embodiment of electronic circuitry 102. In FIG. 2, the electronic circuitry 102 may generally be comprised of: one or more processors 108, such as a Central Processing Unit (CPU) 202; memory 110, such as non-transitory computer-readable medium (e.g., a Random Access Memory (RAM) 204 or alternatively/additionally a storage medium 206 (e.g., read only memory (ROM), hard disk drive, solid state drive, flash memory, cloud storage)); an operating system (OS) 208; one or more application software 210, one or more input devices 212 (hereinafter “input devices”) (e.g., keyboard, mouse, microphone, scanner, camera); and one or more output devices 214 (hereinafter “output devices”) (e.g., LCD screen, LED display, OLED panel). In some embodiments, the input device 212 can further include an analog-to-digital (“A/D”) converter 216, and the output device 214 can further include a digital-to-analog (“D/A”) converter 218. According to some embodiments, the one or more output devices 214 may be a waveform generator or other device to provide an input signal of a known frequency or set of frequencies. According to some embodiments, the one or more input devices 212 and the one or more output devices 214 may be combined in a single device, such as one or more touchscreens or one or more communication interfaces (e.g., RS232, Ethernet, Wifi, Bluetooth, USB). The OS 208 and the one or more application software 210 may be stored in the RAM 204 and/or the storage medium 206. The components of the electronic circuitry 102 may be connected directly or indirectly to one or more printed circuit boards (such as a mother board). Examples include, but are not limited to, personal computers, smart phones, laptops, mobile computing devices, tablet PCs, and servers. Multiple computing devices can be operably linked to form a computer network in a manner as to distribute and share one or more resources, such as clustered computing devices and server banks/farms. In some embodiments, the electronic circuitry 102 may be located locally, and in others they may be located remotely, or they may be a combination of local and remote. For example, the signals between the ultrasound transmitter and receiver 104, 106 and the electronic circuitry 102 may be transmitted wirelessly or over a network. One having ordinary skill in the art will understand that a power supply (not pictured) may also be necessary to operate electronic circuitry 102. In some embodiments, the electronic circuitry can be powered by a radio-frequency power amplifier capable of supplying 400 volts; however, other power supplies may also be suitable.

The memory 110 may be accessible by the one or more processors 108 so that the one or more processors 108 may read information from and write information to the memory 110. The memory 110 may store instructions that, when executed by the one or more processors 108, implement one or more embodiments described herein. The memory 110 may be a non-transitory computer readable medium containing a set of instructions thereon, wherein when executed by a processor (such as one or more processors 108), the instructions cause the processor to perform one or more methods discussed herein.

The electronic circuitry 102 may be an apparatus including: one or more processors (such as one or more processors 108); and memory (such as memory 110) accessible by the one or more processors, the memory storing instructions that when executed by the one or more processors, cause the apparatus to perform one or more methods described herein.

The memory 110 may be a non-transitory processor readable medium containing a set of instructions thereon, wherein when executed by one or more processors (such as one or more processors 108), the instructions cause the one or more processors to perform one or more methods described herein.

According to some embodiments, a system 100 having electrical circuitry 102 may be employed to implement the methods of FIGS. 3 and 4, which are described in further detail below.

According to some embodiments, data may be transferred between the electrical components 102 and a computing system (or between the ultrasound transmitter and receiver 104, 106 and a computing system), stored by the computing system, and/or transferred by the computing system to users of the computing system across local area networks (LANs) (e.g., office networks, home networks), wireless networks (e.g. cellular networks, Wi-Fi networks), or wide area networks (WANs) (e.g., the Internet). In one or more embodiments, the computing system may be comprised of numerous servers communicatively connected across one or more LANs and/or WANs. One having skill in the art would appreciate that there are numerous manners in which the computing system could be configured and embodiments of the present disclosure are contemplated for use with any configuration.

According to some embodiments, the systems and methods provided herein may be employed by a user of a system 100 whether connected to a network or not. Similarly, some steps of the methods provided herein may be performed by components and modules of the computing system whether connected or not. While such components/modules may be offline, the data they generate may then be transmitted to the relevant other parts of the computing system once the offline component/module comes online again with the rest of the network (or a relevant part thereof). According to an embodiment of the present disclosure, some of the applications of the present disclosure may not be accessible when not connected to a network; however, a user or a module/component of the computing system itself may be able to compose data offline from the remainder of the system that will be consumed by the system or its other components when the user/offline system component or module is later connected to the system network.

FIG. 3 is a flow chart depicting an exemplary method 300 for detecting stress fractures in a bone of a subject, according to some embodiments using the exemplary system 100 described above. In some embodiments, certain steps of the method of FIG. 3 are computer-implemented steps. The method of FIG. 3 may be implemented using any suitable system or apparatus, such as the electronic circuitry 102 of FIG. 1 and/or FIG. 2. While an order of operations is indicated in FIG. 3 for illustrative purposes, the timing and ordering of such operations may vary where appropriate without negating the purpose and advantages of the examples set forth in detail.

At step 302, an operator can position an ultrasound transmitter 104 on a subject. More specifically, the ultrasound transmitter 104 can positioned above a bone on the exterior of the subject (e.g., the on skin of the subject, or in acoustic contact with the skin of the subject), and at a non-parallel angle to a transverse direction of the bone, which will be discussed in greater detail below with reference to angle θ_{w}. At step 304, the operator can position the ultrasound receiver 106 above a bone on the exterior of the subject (e.g., the on skin of the subject, or in acoustic contact with the skin of the subject) and at a distance from the ultrasound transmitter 104. At step 306, the electronic circuitry 102 can send a first electrical signal to the transmitter 104. At step 308, the ultrasound transmitter 104 can transmit a first ultrasound signal. In some embodiments, the ultrasound transmitter 104 can convert the first electrical signal to the first ultrasound signal. In some embodiments, transmitting ultrasound waves into the bone of a subject can induce SAWs in the cortical surface of the bone. At step 310, the ultrasound receiver 106 can receive a second ultrasound signal resulting from the induced SAWs which, as will be explained below, can be different from the first ultrasound signal. At step 312, in response to receiving the second ultrasound signal, the ultrasound receiver 106 can produce a second electrical signal. In some embodiments, the ultrasound receiver 106 can convert the second ultrasound signal to the second electrical signal. At step 314, the electronic circuitry 102 can detect the presence of a stress fracture based on the second electrical signal.

As to step 314, in some embodiments, the presence of a stress fracture may be detected by comparing the second electrical signal with a previously acquired electrical signal. In some embodiments, the presence of a stress fracture may be detected by comparing the second electrical signal with a baseline signal. In some embodiments, the presence of a stress fracture may be detected by comparing the second electrical signal with an electrical signal from one or more other receivers. As such, if the comparison is dissimilar enough, the system may determine that a stress fracture is likely present.

As to step 314, in some embodiments, the presence of a stress fracture may be detected by processing the first signal and the second signal to obtain a resulting value or signal and performing a comparison of the resulting value or signal to a reference value or to another second signal.

As to step 314, in some embodiments, the presence of a stress fracture may be detected by comparing the second electrical signal with the first electrical signal to determine a coherence value, and then comparing this coherence value to a threshold or baseline coherence value. In some embodiments, the threshold coherence can be assumed to be 1.0, or a value close to 1.0, such as 0.90, 0.95, 0.97, 0.98, or 0.99. In other embodiments, the threshold can be measured on a known, healthy bone prior to strenuous exercise. As such, if the coherence value is below the threshold coherence value (e.g., a coherence value of 0.7 and a threshold coherence value of 1.0), the system may determine that a stress fracture is likely present. This process will be discussed in further detail below.

Other techniques for detecting the presence of a stress fracture based on the second electrical signal in step 314 will become apparent to those of ordinary skill in the art.

FIG. 4 is a flow chart depicting an exemplary method 400 for detecting stress fractures in a bone of a subject, according to some embodiments using the exemplary system 100 described above. In some embodiments, certain steps of the method of FIG. 4 are computer-implemented steps. The method of FIG. 4 may be implemented using any suitable system or apparatus, such as the electronic circuitry 102 of FIG. 1 and/or FIG. 2. While an order of operations is indicated in FIG. 4 for illustrative purposes, the timing and ordering of such operations may vary where appropriate without negating the purpose and advantages of the examples set forth in detail.

At step 402, the electronic circuitry 102 may generate a first electrical signal from a first data signal using, for example, a digital-to-analog converter 216. At step 404, the ultrasound transmitter 104 may convert the first electrical signal into a first ultrasound signal. At step 406, the ultrasound receiver 106 may convert a second ultrasound signal to a second electrical signal. At step 408, the electronic circuitry 102 may generate a second data signal from the second electrical signal using, for example, an analog-to-digital converter 218. At step 410, the electronic circuitry 102 may detect whether a stress fracture is present in the bone of the subject. At step 412, the electronic circuitry 102 may output the determination from step 410. As examples, the output may be provided on a monitor, on an LCD display, or with one or more LED lights indicating a detected condition. As an example, three output conditions may be displayed using LED lights: likely no stress fracture detected, stress fracture possibly detected, or stress fracture most likely detected.

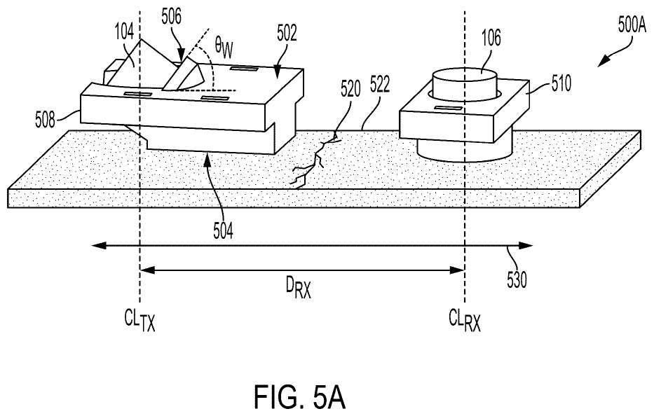

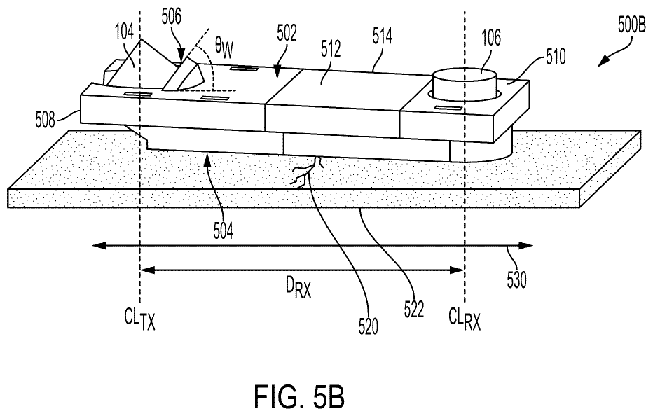

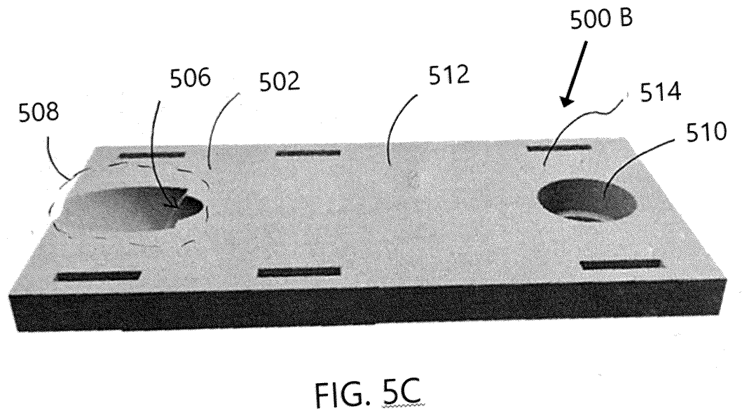

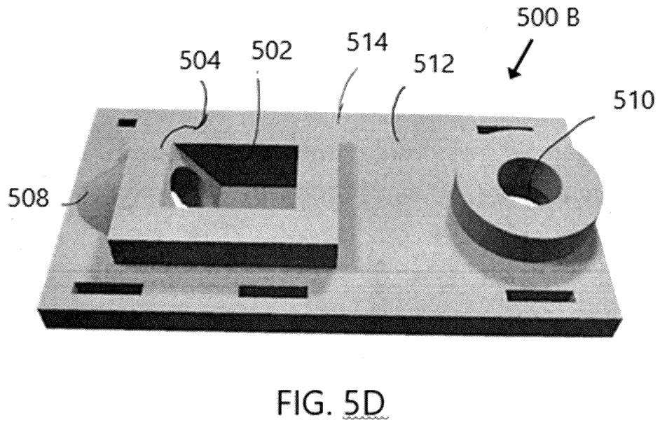

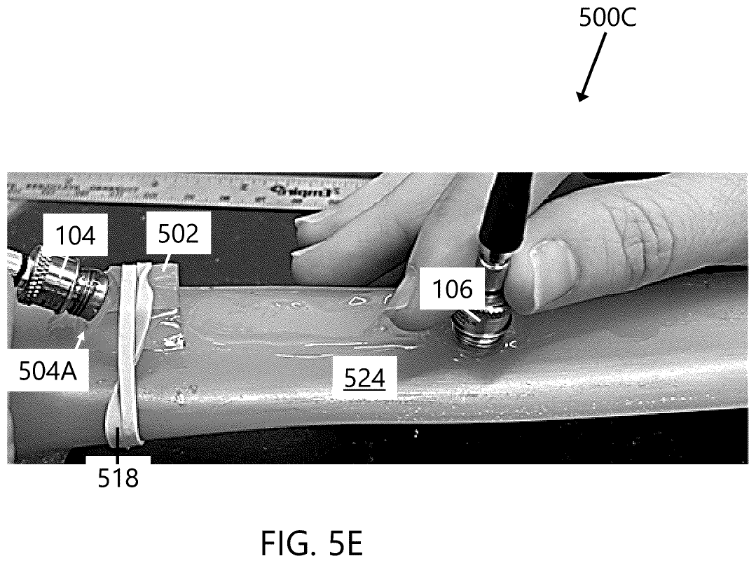

FIGS. 5A-5E depict exemplary apparatuses for detecting fracture or fatigue in a bone of a subject according to some embodiments. Some embodiments disclosed herein can take the form of a handheld device which is portable and capable of being used by non-physician operators in various environments. Particularly, such embodiments can be used on sports fields, military training or operational grounds, or any other environment. To achieve this, some embodiments can include an transmitter mount 502 as is shown in FIGS. 5A-5D. In particular, some embodiments can be a handheld device 500A, 500B for detecting stress fractures 520 in a bone 522 of a subject. According to some embodiments, the handheld device 500A, 500B may include an ultrasound transmitter 104 as described above, an ultrasound receiver 106 as described above, a transmitter mount 502 to hold the ultrasound transmitter 104, and a receiver mount 510 to hold the ultrasound receiver 106.

In some embodiments, for example as shown in FIG. 5A, the transmitter mount 502 can hold the ultrasound transmitter 104, and the receiver mount 510 can hold the ultrasound receiver 106. In some embodiments, for example as shown in FIGS. 5B-5D (wherein FIGS. 5C and 5D depict a top and bottom view, respectively, of the device 500B), an ultrasound mount 514 can include the transmitter mount 502 and the receiver mount 510, which may be connected with a bridging portion 512. In some embodiments, the ultrasound mount 514 may be a unitary structure of the transmitter mount 502, the bridging portion 512, and the receiver mount 510.

As shown in FIG. 5A, with the handheld device 500A, the distance D_{Rx} between the ultrasound transmitter 104 and the ultrasound receiver 106 may vary since the transmitter mount 502 and the receiver mount 510 are not in fixed positions with respect to each other, and such an embodiment may be useful in certain diagnostic situations for a subject. FIG. 5E depicts an experimental setup 500C having similar functionality to the embodiment depicted in FIG. 5A. In particular, the experimental setup shown in FIG. 5E includes an ultrasound transmitter 104 pressed against a simulated contact surface 504A, an ultrasound receiver 106, and a simulated human leg bone 524. Like embodiment 500A, the ultrasound transmitter 104 and the ultrasound receiver 106 of experimental setup 500C are capable of being positioned at different distances from each other.

As shown in FIG. 5B, with the handheld device 500B, the distance D_{Rx} between the ultrasound transmitter 104 and the ultrasound receiver 106 may be fixed since the transmitter mount 502 and the receiver mount 510 are in fixed positions with respect to each other, and such an embodiment may be useful in certain diagnostic situations for a subject.

In some embodiments, the transmitter mount 502, the receiver mount 510, and/or the ultrasound mount 514 can include a handle and/or have a form factor which fits comfortably in the hand of an operator.

In some embodiments, the ultrasound transmitter 104 may be fixed or removably fixed in the transmitter mount 502. In some embodiments, the ultrasound receiver 106 may be fixed or removably fixed in the receiver mount 510.

In some embodiments, such as shown in FIGS. 5A-5D, the transmitter mount 502 can include a contact surface 504, a mounting surface 506, and a transmitter holder 508. The contact surface 504 may be the surface of the transmitter mount 502 which touches the user's skin (e.g., the shin) and may be referred to as the “bottom surface” of the transmitter mount 502. When in use, the contact surface 504 can be held in acoustic contact with the exterior of the subject (and above the bone) to facilitate the transmission of ultrasound signals from the ultrasound transmitter 104 into the bone of the subject. To that end, in some embodiments, the contact surface 504 can have either a flat surface or a concave surface which generally conforms to the shape of the exterior of the subject above the bone (e.g., on the skin above the tibia).

The mounting surface 506 may be an angled wedge surface against which the ultrasound transmitter 104 is situated. The mounting surface 506 may be angled at a non-parallel angle θ_{w} relative to the transverse direction 530 of the bone.

The transmitter holder 508 may be a mechanism that holds or secures the ultrasound transmitter 104 against the mounting surface 506, either in a fixed position or a removably fixed position. In some embodiments, the transmitter holder 508 may include press fit tabs or arms the extend beyond or wrap around the mounting surface 506. By having the ultrasound transmitter 104 removably fixed in the transmitter mount 502, commercially available ultrasound transmitters 104 may interchangeably be used. The transmitter holder 508 can hold the transmitting surface of the ultrasound transmitter 104 at the angle θ_{w} and against the mounting surface 506. The transmitting surface of the ultrasound transmitter 104 may be in acoustic contact with the exterior of the subject (and above the bone).

In some embodiments, the transmitter holder 508 may be secured or held against the subject in a removably fixed position using a holding apparatus. For example, a holding apparatus for the transmitter holder 508 may be an elastic strap, a strap which can be tightened with a buckle or a hook and loop closure, or an adhesive. As an example, in FIG. 5E, the transmitter mount 502 is held in place with an elastic strap 518. In some embodiments, the holding apparatus for the transmitter mount 502 may be integrated with the transmitter mount 502. For example, a strap for securing the transmitter mount 502 to the subject may be permanently attached to the transmitter mount 502.

In some embodiments, the transmitter mount 502 and the ultrasound transmitter 104 may be a unitary body. In some embodiments, the receiver mount 510 and the ultrasound receiver 106 may be a unitary body.

With the handheld device 500A, 500B, the ultrasound transmitter 104 can be held above a bone on the exterior of the subject, and at a non-parallel angle to a transverse direction 530 of the bone (e.g. the tibia). In FIGS. 5A and 5B, for illustration purposes only, the handheld device 500A, 500B is shown directly on the example bone. However, in use, the handheld device 500A, 500B is situated exterior to the subject and in acoustic contact with the skin of the subject. In some embodiments, an acoustically conductive gel may be used to make an acoustic contact between the ultrasound receiver 106 and the skin of the subject and between the ultrasound transmitter 104 and the skin of the subject. In some embodiments, any stress fracture 520 to be detected by the system should be between the ultrasound transmitter 104 and the ultrasound receiver 106. In some embodiments, the handheld device 500A, 500B may be translated or slid down the leg to detect any stress fractures in the subject.

To receive the second ultrasound signals, the ultrasound receiver 106 can be held at a distance D_{Rx} from the ultrasound transmitter 104 along the transverse direction 530 of the bone, and the receiving surface of the ultrasound receiver 106 may be parallel to transverse direction 530 of the bone and in acoustic contact with the exterior of the subject (and above the bone). Distance D_{Rx} can be measured from a centerline CL_{Tx} of the ultrasound transmitter 104 to a centerline CL_{Rx} of the ultrasound receiver 106. In some embodiments, distance D_{Rx} can be greater than or equal to 20 mm and less than or equal to 80 mm; however, one having skill in the art will recognize that other distances D_{Rx} can be possible without diminishing the strength of the second ultrasound signal.

In some embodiments, the system 100 can include two ultrasound receivers 106 which are aligned in the transverse direction but which are two different distances D_{Rx} away from the ultrasound transmitter 104. In this way, the second ultrasound receiver 106 can also receive the second ultrasound signal transmitted by the ultrasound transmitter 104, thereby generating a third electrical signal. In this way, the accuracy of the stress fracture detection process can be enhanced by allowing a comparison of the signals measured at the two ultrasound receivers 106, as will be explained in further detail below. While not exhaustive, the possible comparisons between the second and third electrical signals could include examining time of arrival, amplitude, coherence, frequency content, and changes in frequency content relative to any of the other metrics.

In some embodiments, to acoustically couple the ultrasound transmitter 104 to the skin of the subject, the transmitter mount 502 can at least be partially made from a material which has an acoustic impedance less than the acoustic impedance of the cortical bone. As will be understood by one having skill in the art, the acoustic impedance of the cortical bone can be calculated as

$$Z = ρC = ( bone density ) * ( speed of sound ) .$$

On the other hand, if the acoustic impedance of the transmitter mount 502 is too high, insufficient acoustic energy will make it from the transducer into the cortical bone. To that end, in some embodiments, the contact surface 504 can at least be partially made from polydimethylsiloxane (“PDMS”). Although the entire contact surface 504 can be made from PDMS, in some embodiments, only the portion of the contact surface 504 through which the transmitted ultrasound waves pass can be made from PDMS.

As will now be explained, a number of variables can be set, measured, and/or adjusted for the embodiments disclosed herein to detect a stress fracture in the bone of a subject. In particular, (i) the frequency of the first ultrasound signal which is transmitted by the ultrasound transmitter 104 and (ii) the angle at which the ultrasound transmitter 104 is held relative to the transverse direction of the bone, can each affect the ability of system 100 to detect stress fractures in the bone.

One having skill in the art will understand that to induce SAWs in the cortical bone of a subject, the ultrasound transmitter 104 can transmit ultrasound signals toward the bone of the subject at a specific range of frequencies. For example, if the frequency is too low, the electronic circuitry 102 will not be able to detect any stress fractures. And if the frequency is too high, the SAWs will attenuate too much to be received by the ultrasound receiver 106 at certain distances; as will be understood by one having ordinary skill in the art, the necessary distance can be inversely related to the frequency. Stated another way, selecting the frequency is a balance between (i) sensing resolution/precision and (ii) sensing distance/coverage area. Therefore, in some embodiments, the frequency of the ultrasound signals transmitted by the ultrasound transmitter 104 can be greater than or equal to 1.5 MHz and less than or equal to 3 MHz. In some embodiments, the frequency of the ultrasound signals transmitted by the ultrasound transmitter 104 can be greater than or equal to 0.5 MHz and less than or equal to 5 MHz. In some embodiments, the frequency of the ultrasound signals transmitted by the ultrasound transmitter 104 can be 0.5, 1.0, 1.5, 2.0, 2.5, 3.0, 3.5, 4.0, 4.5, or 5.0 MHz, or any values therebetween.

Similarly, one having skill in the art will also understand that to induce SAWs in the cortical bone of a subject, the ultrasound transmitter 104 can transmit ultrasound signals toward the subject's bone at a specific range of angles θ_{w}, which may be referred to as the “critical angle,” relative to the transverse direction of the bone. One having skill in the art will also understand that SAWs result from a combination of two types of stress waves: longitudinal waves and shear waves. If the angle θ_{w} is too small, the ultrasound transmitter 104 will only produce longitudinal waves in the subject's bone. As the angle θ_{w} increases, the longitudinal waves become shear waves. As angle θ_{w} increases past the critical angle, which for a PDMS wedge and the cortical bone combination may be, for example, 36 degrees, longitudinal and shear waves are no longer produced in the bone, and the surface acoustic waves are produced instead. And if the angle θ_{w} is too large, the ultrasound transmitter 104 may not produce any form of ultrasonic waves (longitudinal, shear, or surface acoustic waves) in the subject's bone. Therefore, in some embodiments, the angle θ_{w} of the ultrasound transmitter 104 (and mounting surface 506) can be greater than or equal to 40 degrees and less than or equal to 65 degrees in order to produce surface acoustic waves for this application. In some embodiments, selecting the material of ultrasound mount 102 to be PDMS, and having an θ_{w} of 55 degrees, may optimize the amount of energy in the surface acoustic wave produced for the relevant distances. In some embodiments, θ_{w} can be 40, 45, 50, 55, 60, or 65 degrees, or any values therebetween.

As explained above, the presence of a stress fracture in a subject can be detected by comparing statistical values related to (i) electrical signals relating to and/or based on the first and second ultrasound signals with (ii) a known threshold value. According to some embodiments, the comparisons may be between the ultrasound signals of a transmitter Tx and a receiver Rx. According to some embodiments, the comparisons may be between the ultrasound signal of the receiver Rx at an unaffected location, and the ultrasound signal of the receiver Rx at the affected (injured) location. According to some embodiments, the comparisons may be between the ultrasound signal of the receiver Rx before the injury (baseline) and after the injury or effect. According to some embodiments, the comparisons may be between the ultrasound signals of two receivers Rx's. According to some embodiments, the comparisons may be coherence comparisons and/or delay time comparisons. As will be understood by one having ordinary skill in the art, other techniques for comparing signal values to obtain statistical values may be possible.

As will be understood by one having skill in the art, “coherence” or “magnitude-squared coherence” may refer to a relationship or correlation between two signals, often baseline and affected signals, which can be calculated as follows:

$${C}_{xy} ( f ) = \frac{{\vert {P}_{xy} ( f ) \vert}^{2}}{{P}_{xx} ( f ) {P}_{yy} ( f )}$$

where C_{xy}(f) represents the magnitude-squared coherence, P_{xy}(f) represents the cross-spectral density between the baseline and affected signals (e.g., altered, perturbed, or modified signals), Pₓₓ(f) represents the frequency of the baseline signal, and P_{yy}(f) represents the frequency of the affected signal. Coherence may be a value between 0.00 (no coherence) and 1.00 (complete coherence). For the application of detecting stress fractures in a bone, the baseline signal can correspond to the ultrasound signal transmitted by ultrasound transmitter 104, and the affected signal can correspond to the ultrasound signal received by ultrasound receiver 106. Once the coherence value is determined, the coherence value can be provided to the user for purposes of detecting the presence of a stress fracture. A coherence of 1.00 can theoretically represent a bone without any fracture, although in practice the coherence of such a bone can be less than 1.00 due to noise, interference, and/or environmental factors; regardless of whether the coherence is less than 1.00 or exactly 1.00, this coherence value can serve as a coherence threshold value C1 to determine the presence of a stress fracture. A coherence of 0.00 can represent a complete bone fracture between the ultrasound transmitter 104 and ultrasound receiver 106. And a coherence between 0.00 and 1.00 (or whatever the coherence threshold value C1 may be) can represent partial fracture, with the fracture size decreasing as the coherence increases and approaches the coherence threshold value C1. According to some embodiments, one or more additional coherence threshold values C2, C3, C4, etc. between 0.00 and C1 may be included to indicate, for example, severity of a stress fracture, depth of a stress fracture, and/or confidence in detecting at least one stress fracture.

In some embodiments, as an example of an output to provide with three LEDs for output devices 214, a first LED may indicate no stress fracture was detected if the coherence is between 1.00 and C1, a second LED may indicate a possible stress fracture was detected if the coherence is between C1 and C2, and a third LED may indicate a very likely stress fracture was detected if the coherence is between C2 and 0.00. For example only and not limitation, in some embodiments, C1 may be a coherence of at least 0.60 and at most 0.80. In some embodiments, C2 may be a coherence of at least 0.30 and at most 0.60.

### Experimental Results

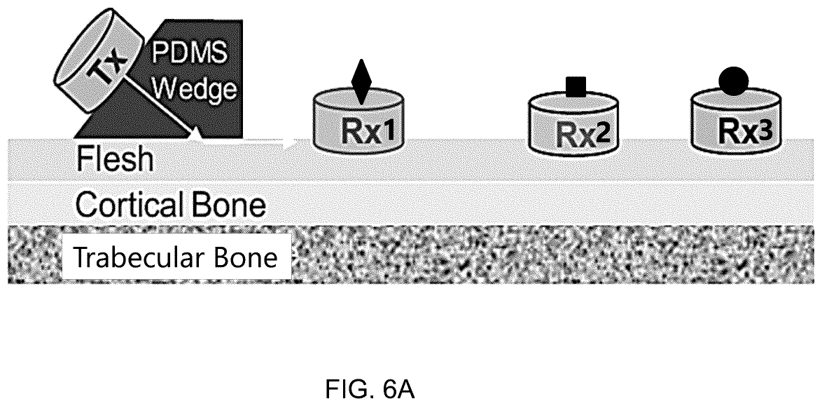

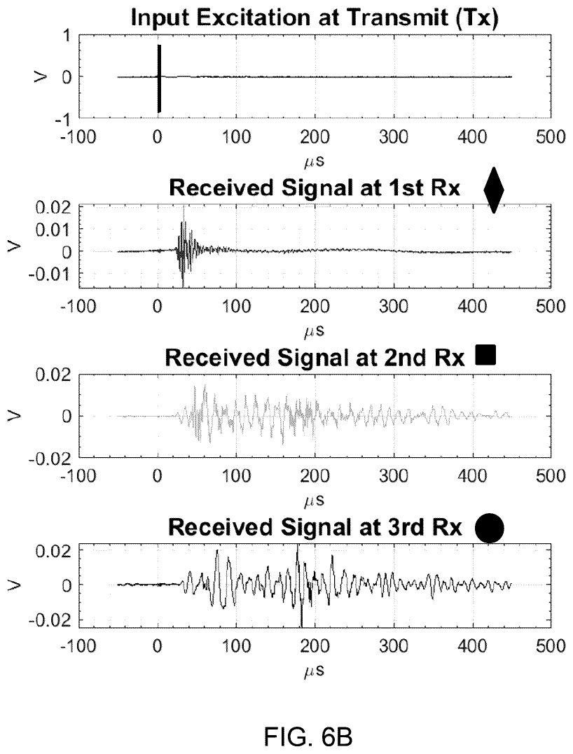

Several experiments were performed according to some embodiments disclosed herein. Reference is now made to those experiments and FIGS. 6A-8D, which depict the results of those experiments. In conducting these experiments, the inventors utilized both commercially-available, artificial tibia bones and cadaveric tibia bones with flesh and skin still attached (also known as post-mortem human subject, or “PMHS,” bones).

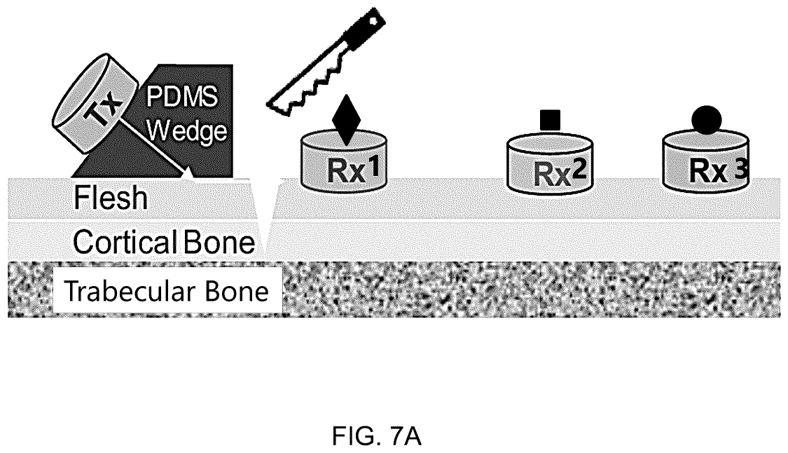

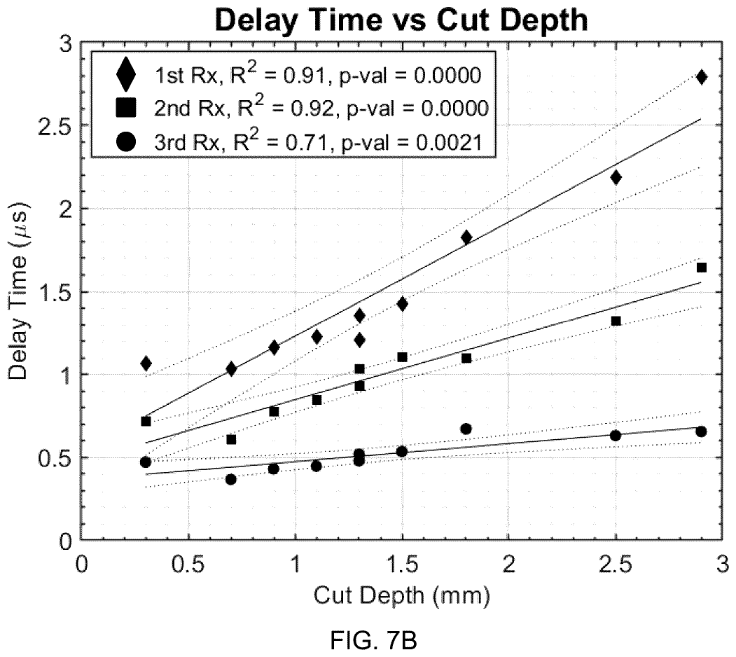

In a first experiment, the inventors measured a threshold delay time for a number of ultrasound receivers positioned at different distances D_{Rx} from the ultrasound transmitter Tx, using a healthy, unfractured bone. FIG. 6A depicts the test setup for this experiment. As can be seen, Rx1, Rx2, and Rx3 represent three ultrasound receivers positioned at different distances D_{Rx} from the ultrasound transmitter. These distances were approximately 35 mm for Rx1, 55 mm for Rx2, and 75 mm for Rx3, in the data shown in FIG. 7B. FIG. 7B depicts the delay time for receiving the signal from the ultrasound transmitter Tx. As can be seen, the results indicate that in a healthy bone, decreasing the distance D_{Rx} results in decreased delay times, and that increasing the distance D_{Rx} decreases the amplitude of the signal seen, as well as temporally spreading out the excited signal.

In a second experiment, the inventors measured the delay time for a number of ultrasound receivers positioned at different distances D_{Rx} from the ultrasound transmitter, using a bone with simulated stress fractures of varying severities (represented by “cut depths”). FIG. 7A depicts the test setup for this experiment. As can be seen, Rx1, Rx2, and Rx3 represent three ultrasound receivers positioned at different distances D_{Rx} from the ultrasound transmitter Tx. These distances for the data in FIG. 7B are approximately 35 mm for receiver sensor Rx1, 55 mm for receiver sensor Rx2, and 75 mm for receiver sensor Rx3. FIG. 7B depicts the delay time for receiving the signal from the ultrasound transmitter Tx, as a function of cut depth. As can be seen, the results indicate that in a fractured bone, both (i) decreasing the distance D_{Rx} and (ii) increasing the cut depth results in increased delay times. Delay time is calculated as the delay between the baseline receive and the affected receive signal (not between the transmit and the receive). For example, Rx1 (the shortest Drx distance), the delay times increase with cut depth, as expected, but when we compare the delay times seen from Rx3 (the longest Drx distance), there is more attenuation and geometric spread of the waveform over the longer distance seen, so less delay time difference is seen between baseline and affected. Notably, increased cut depth corresponds to increased fracture size; therefore, delay times are calculated relative to healthy—or 0 mm—cut depth. The resulting data shows that any of the three receiver sensors (Rx1, Rx2, Rx3) result in a linear relationship between cut depth and delay time, demonstrating that a single Rx at any of the given distance DRx can be used. For this experiment, the greatest change in delay time with cut depth (line slope) is seen with receiver sensor Rx1. As such, the receiver sensor Rx1, which was closer to the ultrasound transmitter Tx than the receiver sensors Rx2 and Rx3, at the smallest DRx, was the most sensitive to detecting small injuries or small cut depths. As such, the receive sensor Rx1 had greater sensitivity compared to receiver sensors Rx2 and Rx3. On the other hand, a healthy bone which is subject to the experiment might have no or virtually no delay time for the signals received at receiver sensors Rx1, Rx2, and Rx3.

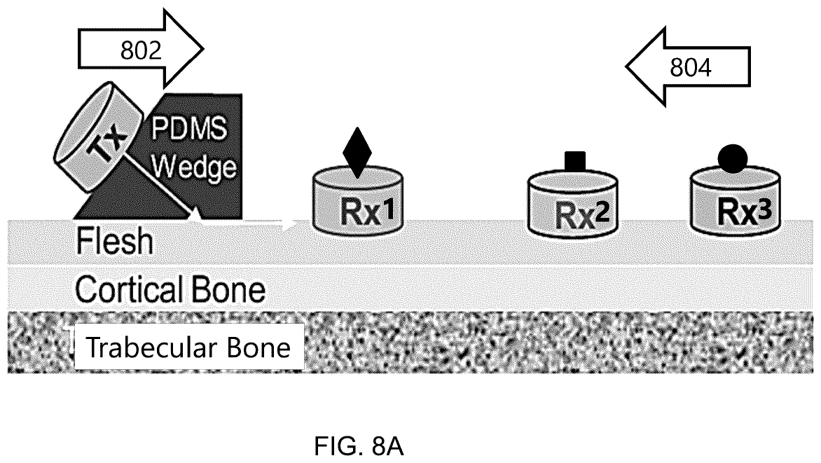

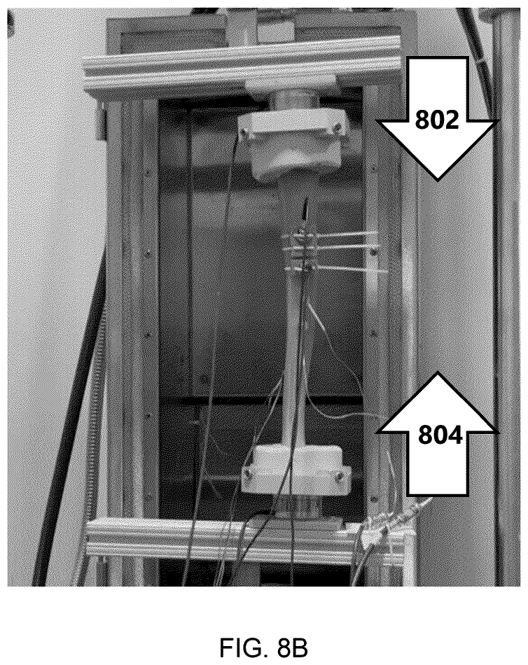

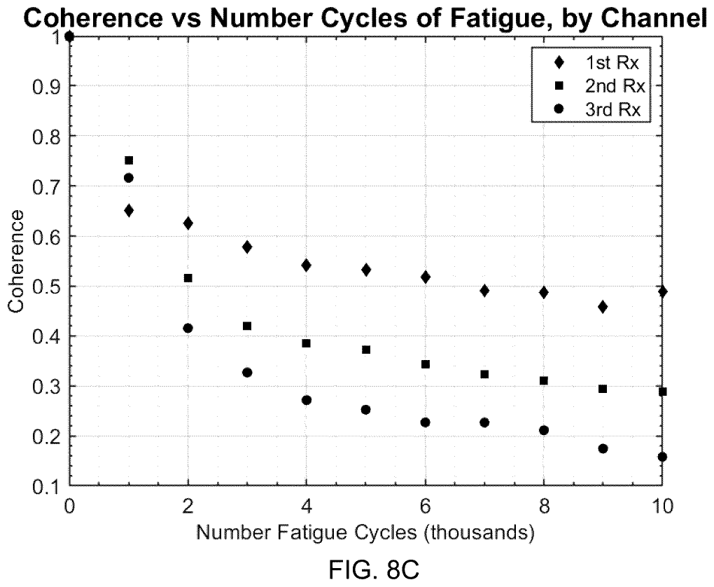

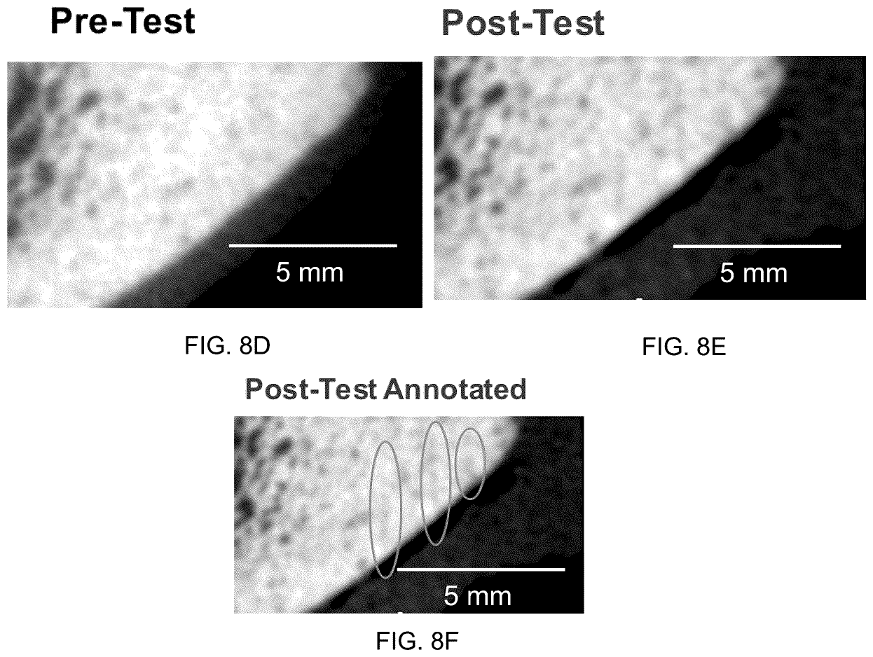

In a third experiment, the inventors measured the delay time and coherence for a number of ultrasound receivers positioned at different distances D_{Rx} from the ultrasound transmitter, while applying a cyclic compression load which simulates running. FIGS. 8A-8B depict the test setup for this experiment. As can be seen, Rx1, Rx2, and Rx3 represent three ultrasound receivers positioned at different distances D_{Rx} from the ultrasound transmitter. Loading arrows 802, 804 represent the direction in which the compression load was applied. As shown in FIG. 8A, the distances D_{Rx} for this experiment are 35 mm, 55 mm, and 75 mm for receiver sensors Rx1, Rx2, and Rx3 respectively. To simulate running, the bone was positioned within a universal testing machine (i.e., universal tension compression machine) and subject to approximately 1,200 N load and 8 mm of displacement over 10,000 fatigue cycles (i.e., steps) at 1.5 Hz. FIG. 8C depicts the coherence at the three ultrasound receivers as a function the number of fatigue cycles, and stress fractures begin to develop. As can be seen with reference to Rx3, when the number of fatigue cycles is zero, the coherence is 1.00. As expected, all three receivers experience a decrease in coherence as the number of fatigue cycles approaches 10,000. Finally, FIGS. 8D, 8E, and 8F depict “before” and “after” microCT images of the bone, which confirm that at least three microfractures developed during the 10,000 cycle test process. FIG. 8D depicts the microCT image of the bone before the test was performed. As can be seen, no stress fractures exist. FIG. 8E depicts the microCT image of the same portion of the bone after the conclusion of the test (i.e., after 10,000 fatigue cycles, and FIG. 8F depicts an annotated version of the microCT image of FIG. 8E. As can be seen, at least three microfractures developed from the test, which were detected using the invention.

### Illustrative Embodiments

The invention includes other illustrative embodiments (“Embodiments”) as follows.

Embodiment 1. A system for detecting fracture or fatigue in a bone of a subject, the system comprising: an ultrasound transmitter capable of being located exterior to the subject; an ultrasound receiver capable of being located exterior to the subject; and electronic circuitry to perform a method, the method comprising: sending a first signal to the ultrasound transmitter, the first signal configured to cause the ultrasound transmitter to transmit a first ultrasound signal; receiving a second signal from the ultrasound receiver, the ultrasound receiver producing the second signal in response to a second ultrasound signal received by the ultrasound receiver; and processing the first signal and the second signal to detect fracture or fatigue in the bone of the subject.

Embodiment 2. The system of embodiment 1, wherein the first ultrasound signal has a frequency greater than or equal to 0.5 MHz and less than or equal to 5.0 MHz.

Embodiment 3. The system of embodiment 1, wherein processing the first signal and the second signal to detect fracture or fatigue in the bone of the subject comprises comparing a statistical value based on the first signal and the second signal to a threshold.

Embodiment 4. The system of embodiment 1, wherein processing the first signal and the second signal to detect fracture or fatigue in the bone of the subject comprises determining coherence between the first signal and the second signal.

Embodiment 5. The system of embodiment 1, wherein processing the first signal and the second signal to detect fracture or fatigue in the bone of the subject comprises determining delay time between the first signal and the second signal.

Embodiment 6. The system of embodiment 1, wherein processing the first signal and the second signal to detect fracture or fatigue in the bone of the subject comprises: determining magnitude-squared coherence between the first signal and the second signal; and providing an output indicating detection of fracture or fatigue in the bone of the subject when the magnitude-squared coherence is greater than or equal to a threshold.

Embodiment 7. The system of embodiment 1, wherein: the first ultrasound signal is capable of inducing surface acoustic waves in the bone when the ultrasound transmitter is placed at an angle to a transverse direction of the bone and not parallel to the transverse direction of the bone; and the second ultrasound signal is capable of being produced in response to the first ultrasound signal when the ultrasound receiver is placed on skin in proximity of the bone of the subject and placed a distance from the ultrasound transmitter.

Embodiment 8. The system of embodiment, further comprising a second ultrasound receiver configured to receive the second ultrasound signal and produce a third signal in response, wherein the third signal is processed with the first signal and the second signal to enhance detection of the at least one of fracture or fatigue in the bone of the subject.

Embodiment 9. An apparatus for detecting fracture or fatigue in a bone of a subject, the apparatus comprising: an ultrasound transmitter configured to transmit a first ultrasound signal; a transmitter mount having a contact surface, a mounting surface, and a transmitter holder, wherein: the contact surface is configured to contact a surface of the subject; the mounting surface is angled at an angle to a transverse direction of the bone and not parallel to the transverse direction of the bone; and the transmitter holder is configured to hold the ultrasound transmitter proximate the subject and at the angle; and an ultrasound receiver configured to receive a second ultrasound signal and positioned at a distance from the ultrasound transmitter.

Embodiment 10. The apparatus of embodiment 9, wherein the angle is greater than or equal to 40 degrees.

Embodiment 11. The apparatus of embodiment 9, wherein the distance is greater than or equal to 20 mm and less than or equal to 80 mm when measured from a centerline of the ultrasound transmitter to a centerline of the ultrasound receiver.

Embodiment 12. The apparatus of embodiment 9, wherein the apparatus is further configured to hold the ultrasound receiver proximate the subject and parallel to the contact surface.

Embodiment 13. The apparatus of embodiment 9, wherein the transmitter mount comprises a material having an acoustic impedance less than an acoustic impedance of a cortical bone.

Embodiment 14. The apparatus of embodiment 9, wherein the ultrasound mount is configured to hold the ultrasound transmitter exterior to the subject.

Embodiment 15. The apparatus of embodiment 9, wherein the apparatus is further configured to hold the ultrasound receiver proximate the subject and exterior to the subject.

Embodiment 16. A method for detecting fracture or fatigue in a bone of a subject, the method comprising: positioning an ultrasound transmitter proximate to the subject, exterior to the subject, and above the bone of the subject, the ultrasound transmitter being positioned at an angle to a transverse direction of the bone and not parallel to the transverse direction of the bone; positioning an ultrasound receiver proximate the subject, exterior to the subject, and above the bone of the subject, the ultrasound receiver being positioned a distance from the ultrasound transmitter along the transverse direction of the bone; transmitting, from the ultrasound transmitter, first ultrasound signal; receiving, at the ultrasound receiver, second ultrasound signal; and detecting fracture or fatigue in the bone of the subject based on the second ultrasound signal.

Embodiment 17. The method of embodiment 16, wherein the first ultrasound signal has a frequency greater than or equal to 0.5 MHz and less than or equal to 5.0 MHz.

Embodiment 18. The method of embodiment 17, wherein the angle is greater than or equal to 40 degrees.

Embodiment 19. The method of embodiment 18, wherein the distance is greater than or equal to 20 mm and less than or equal to 80 mm when measured from a centerline of the ultrasound transmitter to a centerline of the ultrasound receiver.

Embodiment 20. The method of embodiment 16, wherein transmitting the first ultrasound signal induces surface acoustic waves in the bone of the subject.

The detailed description explains embodiments of the disclosure, together with advantages and features, by way of example with reference to the drawings.

It should also be noted that the terms “first”, “second”, “third”, “upper”, “lower”, and the like may be used herein to modify various elements. These modifiers do not imply a spatial, sequential, or hierarchical order to the modified elements unless specifically stated.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the disclosure is provided in detail in connection with only a limited number of embodiments, it should be readily understood that the disclosure is not limited to such disclosed embodiments. Rather, the disclosure can be modified to incorporate any number of variations, alterations, substitutions or equivalent arrangements not heretofore described, but which are commensurate with the spirit and scope of the disclosure. Additionally, while various embodiments of the disclosure have been described, it is to be understood that the exemplary embodiment(s) may include only some of the described exemplary aspects. Accordingly, the disclosure is not to be seen as limited by the foregoing description but is only limited by the scope of the appended claims.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention need not be limited to the described embodiments, but that it has the full scope defined by the language of the following claims, and equivalents thereof.

What is claimed is:

1. A system for detecting fracture or fatigue in a bone of a subject, the system comprising:
an ultrasound transmitter capable of being located exterior to the subject;
an ultrasound receiver capable of being located exterior to the subject; and
electronic circuitry to perform a method, the method comprising:
sending a first signal to the ultrasound transmitter, the first signal configured to cause the ultrasound transmitter to transmit a first ultrasound signal;
receiving a second signal from the ultrasound receiver, the ultrasound receiver producing the second signal in response to a second ultrasound signal received by the ultrasound receiver; and
processing the first signal and the second signal to detect at least one of fracture or fatigue in the bone of the subject.

2. The system of claim 1, wherein the first ultrasound signal has a frequency greater than or equal to 0.5 MHz and less than or equal to 5.0 MHz.

3. The system of claim 1, wherein processing the first signal and the second signal to detect at least one of fracture or fatigue in the bone of the subject comprises comparing a statistical value based on the first signal and the second signal to a threshold.

4. The system of claim 1, wherein processing the first signal and the second signal to detect at least one of fracture or fatigue in the bone of the subject comprises determining coherence between the first signal and the second signal.

5. The system of claim 1, wherein processing the first signal and the second signal to detect at least one of fracture or fatigue in the bone of the subject comprises determining delay time between the first signal and the second signal.

6. The system of claim 1, wherein processing the first signal and the second signal to detect at least one of fracture or fatigue in the bone of the subject comprises:
determining magnitude-squared coherence between the first signal and the second signal; and
providing an output indicating detection of at least one of fracture or fatigue in the bone of the subject when the magnitude-squared coherence is greater than or equal to a threshold.

7. The system of claim 1, wherein:
the first ultrasound signal is capable of inducing surface acoustic waves in the bone when the ultrasound transmitter is placed at an angle to a transverse direction of the bone and not parallel to the transverse direction of the bone; and
the second ultrasound signal is capable of being produced in response to the first ultrasound signal when the ultrasound receiver is placed on skin in proximity of the bone of the subject and placed a distance from the ultrasound transmitter.

8. The system of claim 1, further comprising a second ultrasound receiver configured to receive the second ultrasound signal and produce a third signal in response, wherein the third signal is processed with the first signal and the second signal to enhance detection of the at least one of fracture or fatigue in the bone of the subject.

9. An apparatus for detecting fracture or fatigue in a bone of a subject, the apparatus comprising:
an ultrasound transmitter configured to transmit a first ultrasound signal;
a transmitter mount having a contact surface, a mounting surface, and a transmitter holder, wherein:
the contact surface is configured to contact a surface of the subject;
the mounting surface is angled at an angle to a transverse direction of the bone and not parallel to the transverse direction of the bone; and
the transmitter holder is configured to hold the ultrasound transmitter proximate the subject and at the angle; and
an ultrasound receiver configured to receive a second ultrasound signal and positioned at a distance from the ultrasound transmitter.

10. The apparatus of claim 9, wherein the angle is greater than or equal to 40 degrees.

11. The apparatus of claim 9, wherein the distance is greater than or equal to 20 mm and less than or equal to 80 mm when measured from a centerline of the ultrasound transmitter to a centerline of the ultrasound receiver.

12. The apparatus of claim 9, wherein the apparatus is further configured to hold the ultrasound receiver proximate the subject and parallel to the contact surface.

13. The apparatus of claim 9, wherein the transmitter mount comprises a material having an acoustic impedance less than an acoustic impedance of a cortical bone.

14. The apparatus of claim 9, wherein the transmitter mount is configured to hold the ultrasound transmitter exterior to the subject.

15. The apparatus of claim 9, wherein the apparatus is further configured to hold the ultrasound receiver proximate the subject and exterior to the subject.

16. A method for detecting fracture or fatigue in a bone of a subject, the method comprising:
positioning an ultrasound transmitter proximate to the subject, exterior to the subject, and above the bone of the subject, the ultrasound transmitter being positioned at an angle to a transverse direction of the bone and not parallel to the transverse direction of the bone;
positioning an ultrasound receiver proximate the subject, exterior to the subject, and above the bone of the subject, the ultrasound receiver being positioned a distance from the ultrasound transmitter along the transverse direction of the bone;
transmitting, from the ultrasound transmitter, first ultrasound signal;
receiving, at the ultrasound receiver, second ultrasound signal; and
detecting at least one of fracture or fatigue in the bone of the subject based on the second ultrasound signal.

17. The method of claim 16, wherein the first ultrasound signal has a frequency greater than or equal to 0.5 MHz and less than or equal to 5.0 MHz.

18. The method of claim 17, wherein the angle is greater than or equal to 40 degrees.

19. The method of claim 18, wherein the distance is greater than or equal to 20 mm and less than or equal to 80 mm when measured from a centerline of the ultrasound transmitter to a centerline of the ultrasound receiver.

20. The method of claim 16, wherein transmitting the first ultrasound signal induces surface acoustic waves in the bone of the subject.
